(19) United States
(12) Patent Application Publication
(10) Pub. No.: US 2009/0257984 A1
(43) Pub. Date: Oct. 15, 2009

(54) ANTIFUNGAL COMPOSITIONS CONTAINING THE ENDOPHYTE FUNGUS ALTERNARIA ALTERNATA AND OR ITS METABOLITES, AS ANTAGONIST AGENTS OF PLASMOPARA VITICOLA

(76) Inventors: Rita Musetti, Udine (IT); Stefano Borselli, Pordenone (IT); Michele D'Ambrosio, Povo (IT)

Correspondence Address: NIXON & VANDERHYE, PC, 901 NORTH GLEBE ROAD, 11TH FLOOR, ARLINGTON, VA, 22203, US

(21) Appl. No.: 12/305,136

(22) PCT Filed: Jun. 14, 2007
(86) PCT No.: PCT/IB2007/052262

§ 371 Date: Apr. 10, 2009

(30) Foreign Application Priority Data
Jun. 16, 2006 (IT) RM2006A000315

(51) Int. Cl. A61K 45/00; A61K 36/06; A61K 31/4985; A61P 31/10

(52) U.S. Cl. 424/93.5; 424/195.15; 514/249

(57) ABSTRACT

The present invention relates to compositions containing the fungus Alternaria alternata and/or the secondary metabolites thereof, which belong to the family of the diketopiperazines (DKPs), for controlling the pathogen fungus Plasmopara viticola.

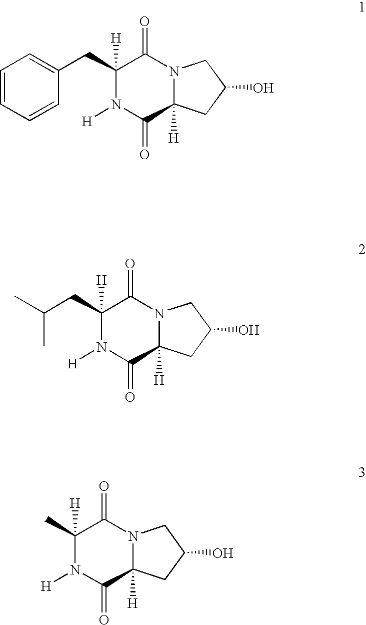

The present invention relates to compositions containing the fungus Alternaria alternata and, or the metabolites thereof, belonging to the family of the diketopiperazines (DKPs), for controlling the pathogen fungus Plasmopara viticola, agent of the vine peronospora. The main three metabolites are the following compounds:

cyclo(L-phenylanine-trans-4-hydroxy-L-proline) (1)

cyclo(L-leucine-trans-4-hydroxy-L-proline) (2)

cyclo(L-alanine-trans-4-hydroxy-L-proline) (3)

having formula, respectively:

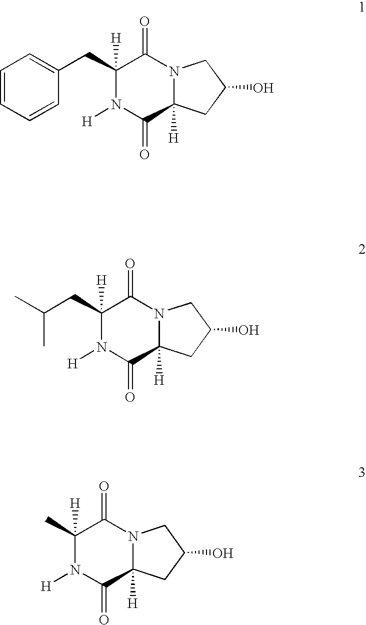

All vine varieties of Vitis vinifera are susceptible to peronospora which is caused by the pathogen Plasmopara viticola. On the contrary, species of Vitis americana, coming from humid regions wherein the parasite's presence has exerted a strong selection onto the local populations, are resistant.

Until the '50s, chemical struggle against the vine peronospora has been based almost exclusively upon the use of coating rameic products: Bordeaux mixture and copper oxychorides which, in addition to the strong activity against peronospora, are endowed with a not negligible activity against Oidium and Botritis. The cupric products have been partially replaced by several synthesis organic products (carbamates, phthalymmidics, etc.). Still nowadays two strategies are mainly followed to limit the damages caused by the pathogen: the continuous coating and the guided struggle.

The proposed solutions can reveal inadequate especially in the periods of continuous rain when one has to resort to emergency treatments by using systemic fungicides which can be applied once the infection has occurred. However, the riskiness of such intervention lies in the possibility of creating resistant strains of the fungus.

Furthermore, from recent studies performed on coating acupric products, it has been underlined that although they do not create phytotoxicity onto the vine, differently from copper, they can be noxious for man. In fact, it has been found that in the workers the vinegrowing and wine producing sectors traces of ETU (ethylene thiourea, the degradation product of Mancozeb) on the average four times higher than those which can be found on the average in the remaining population have been found. It is to be reminded that ethylenethiourea is considered a potentially carcinogen substance and this, on itself, is sufficient to explain the reason of the search for alternative methods.

Taking into account that the struggle strategies only based upon phytoiatric interventions easily produce various drawbacks, synergic combinations of different and complementary struggle methods with partial single efficacy have been searched for. Such methods are generally designated as methods of integrated struggle which are substantially based upon the combined use of low-dosage fungicides and antagonists of the pathogen(s). However, they are not characterized by a complete efficacy. A concrete example of integrated struggle against cryptogram diseases is given by the study performed by Raziq and Fox (2002) upon the integrated control of Armillaria mellea on apple plants, both with antagonists and with fungicides. In the specific case of the P. viticola, patented integrated methods do not yet result to exist.

In biological viticulture the struggle against the vine peronospora has been so far mainly based upon the use of natural substances, copper in particular. However, the use thereof is under discussion subject due to the possible negative after-effects onto the environment, consequent to the accumulation thereof onto the ground. For this reason, the use of cupric compounds, starting as from 2002, has been subjected to specific law limitations in the field of the biological agriculture with the purpose of fixing a maximum value of metal copper which can be distributed.

A real alternative for the defence against the pathogens is represented by the use of antagonist organisms. The antagonist, or biocontrol agent, is an organism existing in nature which can limit the growth of the pathogen and keep it under the threshold of economical damage; the antagonist does not wholly eliminate the pathogen, but it limits the development thereof and, consequently, the damages caused thereby onto the culture.

In literature, there are examples of applications of antagonists for controlling some diseases caused by pathogen organisms onto different vegetable varieties and some of them relate to the vine peronospora (Falk et al., 1996; Kortekamp, 1997). However, for the vine, nothing has been reported as to the use of endophyte fungi as antagonist organisms and, in particular, of Alternaria alternata, and of the metabolites produced thereby.

The endophytes are microrganisms (fungi, bacteria and actinomycets) spending part or the whole biological cycle thereof inside the host plant without causing visible symptoms of disease. The endophyte fungus, then, is “asymptomatic” and the relationship with the plant can be interpreted as an interaction of mutual type (Clay, 1991). Endophytes are able to synthesize a wide range of secondary metabolites which can be widely applied in medicine, in the pharmaceutical industry and in agriculture (Gusman e Vanhaelen, 2000).

It has been now surprisingly found that the endophyte fungus of the vine, Alternaria alternata, and, or some of the secondary metabolites thereof, can be used as antagonists against Plasmopara viticola. Advantageously, the use thereof takes place under the form of compositions containing them together with excipients and additives usually used in antifungal compositions to be applied onto the plants.

The object of the present invention are therefore is antifungal compositions containing the Alternaria alternata fungus in the various forms thereof as antagonist agent of Plasmopara viticola together with excipients and additives suitable for the use in antifungal compositions to be used in agriculture. Advantageously, mycelium of alternated Alternaria in active growth is used.

An additional object of the present invention is a process for producing from Alternaria alternata an antagonist extract for Plasmopara viticola comprising the following steps:
a. inoculation of culture broth with portions of growing mycelium of Alternaria alternata, incubation thereof possibly under stirring for 12-60 hours, freezing and lyophilization;
b. dissolving in water the lyophilized broth obtained from step a), filtering and extraction of the aqueous solution with solvent of average polarity, preferably n-butanol, until obtaining an organic phase;
c. fractionation of said organic phase by means of preparative chromatography on silica layer by eluting with a mixture of n-butanol/acetic acid/water in ponderal ratio 60:15:25 until obtaining three fractions A, B and C;
d. additional fractionation of the fractions A and B by means of chromatography onto a thin layer by eluting with a mixture of dichloromethane/methanol in ponderal ratio 9:1 until obtaining the compounds of the following formulas (1) (R_{f} s0.48) and (2) (R_{f} 0.43)

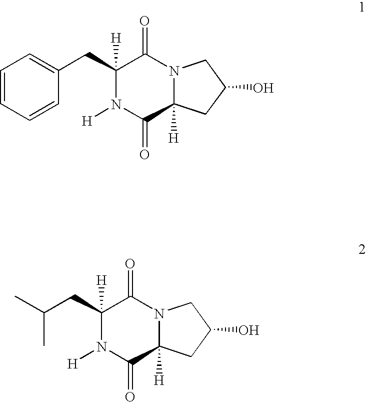

An additional stadium e), still forming subject of the invention, can be added to the process described above, in which stadium said fraction C is subjected to chromatography onto thin layer by eluting with a mixture of dichloromethane/methanol in ponderal ratio 85:15 until obtaining the compound of following formula (3) (R_{f} 0.40)

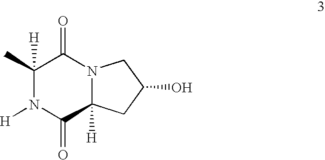

According to the invention antagonist compositions of Plasmopara viticola containing one or more of the three fractions A, B and,or C and,or one or more of the compounds (1), (2) and,or (3) together with additives and,or excipients of common use in the antifungal compositions, used in agriculture, may be produced.

A. alternata has been already signalized as endophyte fungus in several herbaceous and arboreal forest species; up to know signalizings in Vitis vinifera (Cardinali et al., 1994) have been few, but no signalizing has ever related the production of dichetopiperazine (DKPs) by the vine endophyte A. alternata.

The secondary metabolites of Alternaria alternata, the use thereof is proposed according to the present invention, are known compounds belonging to the chemical family of diketopiperazines (DKPs).

The diketopiperazines are small peptide derivatives which can be found in nature both in the animal and vegetable kingdom. They have a simple chemical configuration, they are stable to proteolysis, their synthetic routes are well known, therefore they can be synthesized easily in laboratory (Horton et al., 2000).

In medical field, they are used as antibiotics and synthetic vaccines, as chemotherapeutic products and as antihypertensive agents; in agriculture they have been utilized against abiotic stresses and as antibacterial and antifungal substances (Horton et al., 2000; McCleland et al., 2004), but never signalized against vine pathogens and in particular against P. viticola.

As far as the signalizing relating to other active dipeptide molecules against pathogen fungi, which among Plasmopara viticola is comprised, may be cited: U.S. Pat. Nos. 6,084,065 Camaggi et al and 6,448,228 Filippini et. al. The above mentioned patents disclose the discovery of new synthesis ways for producing peptide molecules with antimicrobic and pesticidal action.

The advantages involved in the use of the fungus in the struggle against Plasmopara viticola are the following ones: the endophyte fungus causes neither symptoms nor other vine alterations, but it is able to alter significantly the pathogen by jeopardizing the sporulation capability; the DKPs extracted directly from the culture broth can be used in biological viticulture, as natural compounds extracted from “natural” organisms.

Hereinafter some examples related to the invention will be reported.

### EXAMPLE 1

126 vine endophyte microrganisms were grown in liquid medium (Nutrient Broth=NB plus sucrose) in 48-well sterile plates, placed onto an oscillating stirrer at 150 orbits/min for 48 hours at room temperature. 900 μl of NB and one kind of microorganism in suspension (suspension 1) were put into each well. For each organism 5 repetitions were set.

Subsequently, small disks cut from healthy vine leaves were placed in contact for 5 minutes with the antagonist suspension and then inoculated into plates with peronospora spores in concentration of 4.25×10⁵ sporangia/ml for one night at 20° C. (suspension 2).

Then, the small disks were placed into humid chamber at 20° C. in thermostat for one week.

At the end, the presence of sporangia of P. viticola was checked, with the help of the stereomicroscope. This test was repeated 6 times during the years 2002 and 2003.

Out of 126 microrganisms, 5 constantly inhibited the sporulation of P. viticola. Thus, they were kept in purity and identified. All five insulated products belong to the Alternaria alternata species.

From the same leaf small disks mentioned above, after 3 and 6 days from inoculation with peronospora, tissue portions were sampled for the analysis with the Transmissione Electron Microscope (TEM), with the purpose of checking the interactions at cellular level among vine, P. viticola and A. alternata.

The samplings and the observations were performed also onto control tissue portions (healthy vine, vine inoculated with A. alternata only and vine inoculated with P. viticola only).

From the observations with TEM it resulted that A. alternata does not induce ultrastructural alterations in the leaf tissues of the vine; in fact, the tissues appear to be well preserved; on the contrary, P. viticola shows serious alterations. The mycelium has few, wide vacuoles which sometimes contain dark precipitates. The haustoria of the oomycetes are aborted. P. viticola was never found between the stoma's guard cells, during the escaping phase.

### EXAMPLE 2

The production of low molecular weight metabolites by the endophyte A. alternata was implemented starting from the fungus' culture broth. Flasks containing 250 ml of Nutrient Broth were inoculated with portions of mycelium in active growth of A. alternata, kept in Petri capsule on agarized medium. The broth was then incubated on a shaker at 20° C., 48 h, then frozen (−40° C.) and lyophilized. Subsequently the lyophilized broth (20 g) was dissolved in water and filtered; then the aqueous solution was extracted with n-butanol. This organic phase was fractionated by means of preparatory layer Chromatography (PLC) onto silica plate (Merck-Kieselgel 60 PF₂₅₄) and eluted with a mixture of n-butanol/acetic acid/H₂O 60:15:25 until obtaining three bands, called A, B and C in ascending order of polarity. The fractions A (54 mg) and B (48 mg) were further purified by means of thin layer chromatography (TLC) onto silica plate and eluted with a dichloromethane and methanol mixture (CH₂Cl₂/MeOH 9:1) to obtain the pure compounds 1 (1.5 mg, R_{f} 0.48) and 2 (5 mg, R_{f} 0.43). The fraction C (83 mg) was subjected to TLC (CH₂Cl₂/MeOH 85:15) too, until obtaining another pure compound, 3 (3.5 mg, R_{f} 0.40). The insulated metabolites were characterized by protonic magnetic resonance spectroscopy (Bruker AV400, spectra ¹H and 2D-NMR at 400 MHz) using as standard the signals of the residual solvent (δ in ppm, CHD₂OD=3.31, CHCl₃=7.26, D₂O=4.90). Mass spectrometry data were obtained by means of spectrometer Kratos MS80. The optical rotations were measured by means of polarimeter JASCO-DIP-181 by using a 10-cm cell. The molecular formula of the main low molecular weight metabolites produced by A. alternata in liquid culture were calculated by means of electronic impact mass spectrometry (Electron Impact Mass Spectrometry, EIMS, and High Resolution EIMS, HREIMS).

Three DKPs were extracted in higher quantity compared to the other ones. They are:

cyclo(L-phenilalanine-trans-4-hydroxy-L-proline) (1)

cyclo(L-leucyne-trans-4-hydroxy-L-proline) (2)

cyclo(L-alanine-trans-4-hydroxy-L-proline) (3)

and have the following chemical formulas:

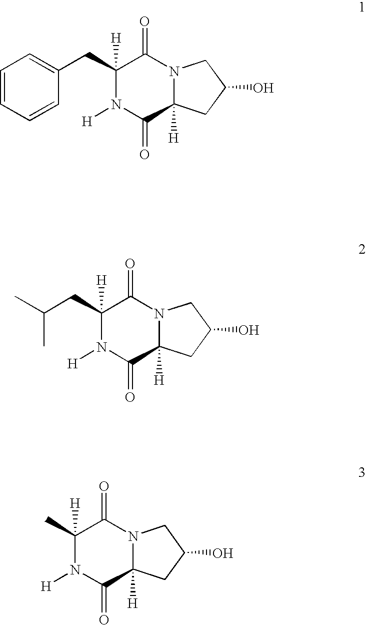

Such metabolites were tested at different concentrations singularly and in combination against P. viticola, both on leaf small disks kept in humid chamber (in vitro) and in plants kept in greenhouse (in planta).

Aqueous solutions were prepared at the following concentrations: 10⁻³ M, 10⁻⁴ M, 10⁻⁵ M and 10⁻⁶ M.

Solutions were prepared also by taking the molecules twice a time and all three of them altogether, by obtaining however always the same end concentrations.

The test on the leaf small disks was performed in the same way as described above.

### EXAMPLE 3

For the in-planta test, rooted cuttings of vines, cultivar Pinot grigio, were used, grown under controlled conditions in greenhouse (20° C., photoperiod 12 h); the cuttings were arranged on polystyrene panels by using staples so that the lower page of the leaves could remain as much adherent as possible to the plane.

The in-planta test was articulated in two experiments:
1) Curative experiment to evaluate the DKPs' efficacy after 2 h and after 24 h as from inoculation of P. viticola;
2) Preventive experiment to evaluate the treatment efficacy before inoculation of P. viticola.

### 1) Curative Test

Each leaf was initialled with a number and a letter based upon the DKP type and the used concentration; whereas onto the lower page, small circles were traced to underline the place wherein the solution containing DKP would have been positioned. Then, the leaves were sprayed with a suspension containing Peronospora sporangia at concentration of 4.7×10⁵ sporangia/ml (except for those destined to the preventive experiment). After a period of 2 h and 24 h from inoculation with the suspension containing Peronospora sporangia, drops of DKP solution (each drop corresponds to 22 μl) were placed in the respective small circles. At the end of the incubation period of the pathogen (7 days), the leaves were observed to evaluate if there was a sporulation by the P. viticola or if it had been inhibited by the treatment with DKPs.

### 2) Preventive Test

Also in this test the leaves were initialled with numbers and letters based upon the DKP type and used concentration, and on the lower page of the leaf small circles were drawn (6 for each leaf) to underline the area thereof wherein the drop of solution containing the DKP molecule would have been positioned. These latter were immediately positioned in the respective small circles. At the end of the incubation period the leaves were observed to evaluate if sporulation by P. viticola had taken place or if it had been inhibited by the DKPs' action.

The DKPs efficacy on the leaf small disks resulted to be good (reduction in the sporulation of P. viticola by 80%), both using DKPs with curative and preventive purpose. The solutions prepared by using the mix of DKPs inhibited the pathogen sporulation by 100%. Also in the in planta test, DKPs inhibited the sporulation of P. viticola both in the curative experiments (after 2 h and after 24 h as from the infection) and in the preventive experiments.

These substances did not provoke necrosis or any other toxic effect in the leaf tissues of V. vinifera; additional cytological surveys further demonstrated that the mix of DKPs (at the concentration 10⁻⁴ M) did not provoke alterations to the cellular structures of the vine leaf tissue; on the contrary, P. viticola, inoculated on the leaf small disks and treated after 2 h with DKPs showed a very altered mycelium. In fact, the vacuoles appeared to be deformed and the haustoria necrotic. Such ultrastructural modifications have already been signalized in other pathogen fungi after treatment with antagonists, by indicating the action of metabolites and/or toxic entymes. In particular, the presence of vacuolar anomalous structures (vacuola with big sizes and in reduced number) inside the mycelium of P. viticola, is correlated with the fungus' senescence phase; senescence which can appear both for physiological reasons (end phase of the pathogen cycle), but also for the presence of antagonists and/or toxic substances (as in the cases reported by us).

### EXAMPLE 4

In order to apply our inventions in vine plants, also kept in full field, we acted following two protocols.

The first one provided for the preparation of an aqueous solution of DKPs at concentration of 10-3M therewith the treatments were performed on vine, by making sure to spray well the green portions.

The second one provided the preparation of culture broth of Alternaria alternata. Such broth was then filtered by means of sterile gauze and utilized to treat the vines both as preventive and curative way. Treatments were made also by means of aqueous suspension of homogenized mycelium of Alternaria alternata.

The results shown here demonstrate that the DKPs extracted from the culture liquid of the endophyte of A. alternata vine, appear to be promising means for controlling P. viticola for different reasons:
1) they have not resulted to be toxic for the vine;
2) they are low molecular weight compounds extremely easy also to be synthesized;
3) the costs of a possible production would be contained;
4) their efficacy level has demonstrated to be very interesting above all in view of the new European law about the additional limitation to the copper use in biological viticulture ((EC) REGULATION NR. 473/2002 OF THE COMMISSION of 15 Mar. 2002).

The use of the endophyte fungus Alternaria alternata as antagonist agent of Plasmopara Viticola and the use of the secondary metabolites thereof, in particular those of formulas (1), (2) and (3), as antagonist substances of Plasmopara viticola, form additional subject of the present invention.

1. An antifungal composition containing the fungus Alternaria alternata in the various forms thereof as antagonist agent of Plasmopara viticola together with excipients and/or additives suitable for antifungal compositions used in agriculture.

2. A process for producing from Alternaria alternata an antagonist extract for Plasmopara viticola comprising:
a) inoculating culture broth with portions of growing mycelium of Alternaria alternata, incubating possibly with stirring for 12-60 hours, freezing and lyophilizing;
b) dissolving in water the lyophilized broth obtained from step a), filtering to provide an aqueous filtrate and extracting said the aqueous filtrate with solvent of average polarity, preferably n-butanol, until an organic phase is obtained;
c) fractionating said organic phase by preparative chromatography on silica layer and eluting with a mixture of n-butanol/acetic acid/water in ponderal ratio 60:15:25 until obtaining three fractions A, B and C; and
d) additionally fractionating the fractions A and B by thin layer chromatography and eluting with a mixture of dichloromethane/methanol in ponderal ratio 9:1 until obtaining the following compounds of formula (1) (R_{f} s0.48) and (2) (R_{f} 0.43)

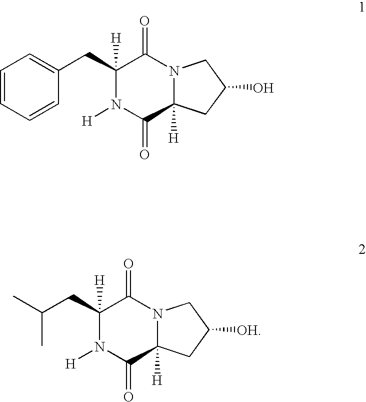

3. The process according to claim 2, further comprising:
e) fractionating said fraction C by thin layer chromatography and eluting with a mixture of dichloromethane/methanol in ponderal ratio 85:15 until obtaining the following compound of formula (3) (R_{f} 0.40)

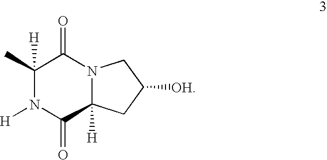

4. An antagonist composition of Plasmopara viticola containing one or more of the fractions A, B and/or C obtainable from c) of claim 2, together with additives and,or excipients suitable for antifungal compositions used in agriculture.

5. An antagonist composition of Plasmopara viticola according to claim 4, containing one or more of the compounds (1), (2) and/or (3) together with additives and/or excipients suitable for antifungal compositions used in agriculture.

6. An antagonist composition of Plasmopara viticola according to claim 5 containing one or more of said fractions A, B and/or C and/or one or more of said compounds (1), (2) and/or (3) suitable for antifungal compositions used in agriculture.

7. A method of using fungus Alternaria alternata as antagonist agent of Plasmopara viticola comprising applying said fungus onto a plant.

8. A method of using one or more of the fractions A, B and/or C obtainable from c) of claim 2 as antagonist agents of Plasmopara viticola comprising applying said one or more of the fractions A, B and/or C onto a plant.

9. A method of using one or more of the compounds (1), (2) and/or (3) as antagonist agents of Plasmopara viticola comprising applying said one or more of the compounds (1), (2) and/or (3) onto a plant.
